(19) United States
(12) Patent Application Publication
(10) Pub. No.: US 2026/0057982 A1
(43) Pub. Date: Feb. 26, 2026

(54) MULTI-DATA FIELD IN CALENDAR VIEW

(71) Applicant: Oura Health Oy, Oulu (FI)

(72) Inventors: Mika Erkkilä, Oulu (FI); Janne Lauri Käki, Helsinki (FI)

(21) Appl. No.: 18/815,633

(22) Filed: Aug. 26, 2024

(51) Int. Cl. G16H 15/00

(52) U.S. Cl. CPC G16H 15/00

(57) ABSTRACT

Methods, systems, and devices for displaying information collected from a user are described. A user interface may be configured to display summary information corresponding to an extended duration via a portion of a device display in a calendar view. In some examples, the user interface may be configured to display information fields corresponding to a subset of the extended duration, and the information may correspond to a category of a set of categories. Additionally, or alternatively, the user interface may be configured to display summary information corresponding to a total for the extended duration, an average for the extended duration, or another summary metric. In some examples, the user interface may be configured to readily cycle between the set of categories, thereby facilitating the display of various types of information to a user, even as the display area of a user device may be limited.

### FIELD OF TECHNOLOGY

The following relates to wearable devices and data processing, including display of multiple data fields in a calendar view.

### BACKGROUND

Some wearable devices may be configured to collect data from users associated with users, including temperature data, heart rate data, and the like. Many users have a desire for more insight regarding their physical health.

### BRIEF DESCRIPTION OF THE DRAWINGS

FIG. 1 illustrates an example of a system that supports display of multiple data fields in a calendar view in accordance with aspects of the present disclosure.

FIG. 2 illustrates an example of a system that supports display of multiple data fields in a calendar view in accordance with aspects of the present disclosure.

FIG. 3, FIG. 4, and FIG. 5 show examples of graphical user interfaces (GUIs) that support display of multiple data fields in a calendar view in accordance with aspects of the present disclosure.

FIG. 6 shows a block diagram of an apparatus that supports display of multiple data fields in a calendar view in accordance with aspects of the present disclosure.

FIG. 7 shows a block diagram of a wearable application that supports display of multiple data fields in a calendar view in accordance with aspects of the present disclosure.

FIG. 8 shows a diagram of a system including a device that supports display of multiple data fields in a calendar view in accordance with aspects of the present disclosure.

FIG. 9 shows a flowchart illustrating methods that support display of multiple data fields in a calendar view in accordance with aspects of the present disclosure.

### DETAILED DESCRIPTION

In some examples, a wearable device may collect physiological data from a user (e.g., via one or more sensors), which may be used to determine and classify health and activity information about the user. The information may be displayed via a display of the wearable device or a user device, which may provide health and activity data and insights to the user. In some cases, however, displaying information related to data collected from the user may be challenging. For example, wearable devices (e.g., wrist-worn devices) and user devices may have limited display areas, which may limit the quantity of information that may be readily displayed to a user. Additionally, displaying health and activity metrics calculated from the data collected from the user over a long duration may be challenging in a limited display area.

In accordance with examples as described herein, a user interface may display summary information corresponding to an extended duration (e.g., year, month, weeks, multiple months) via a portion of a device display in a calendar view. In some examples, the user interface may be configured to display information fields corresponding to a subset of the extended duration (e.g., days, weeks, months), and the information may correspond to a category of a set of categories. Additionally, or alternatively, the user interface may be configured to display summary information corresponding to a total for the extended duration, an average for the extended duration, or another summary metric. In some examples, the user interface may be configured to readily change between the set of categories, thereby facilitating the display of various types of information to a user, even as the display area of a user device may be limited.

Aspects of the disclosure are initially described in the context of systems supporting physiological data collection from users via wearable devices. Aspects of the disclosure are additionally illustrated with respect to graphical user interfaces (GUIs). Aspects of the disclosure are further illustrated by and described with reference to apparatus diagrams, system diagrams, and flowcharts that relate to display of multiple data fields in a calendar view.

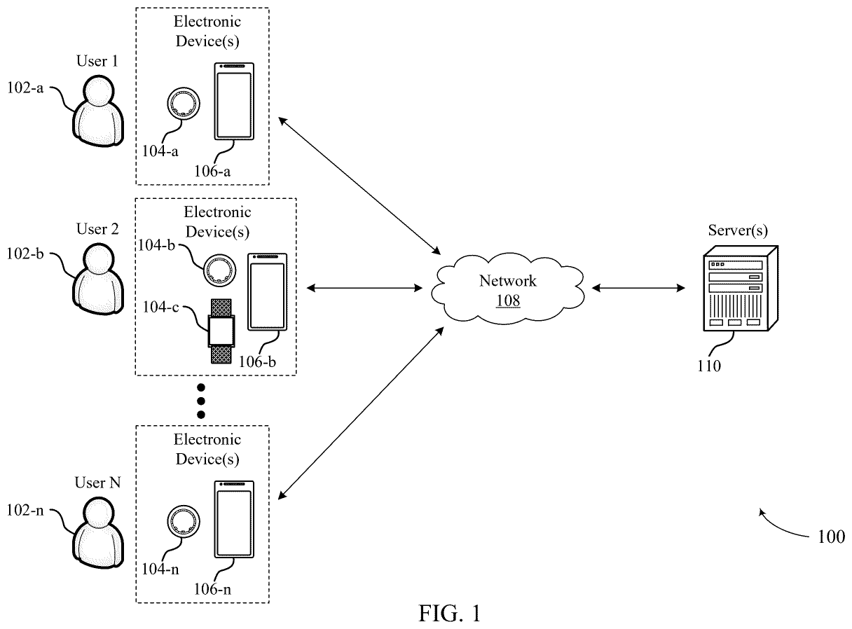

FIG. 1 illustrates an example of a system 100 that supports display of multiple data fields in a calendar view in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some cases, the user devices 106 may display information such as those calculated from data collected by a wearable device 104, including health or activity metrics, sleep data, or other information. In some cases, however, a display size associated with the user devices 106 may be limited, which may hinder the display of information collected from users 102 over some duration. For example, the display of information for a large duration, such as for a month, may be difficult as wearable devices 104 collect various different types of information which may be used to determine various types of metrics and insights, and the information may be difficult to include in a limited display size.

In accordance with examples as described herein, a user interface of a user device 106 (e.g., or a wearable device 104) may be configured to display summary information corresponding to an extended duration (e.g., year, month, weeks, multiple months) via a portion of a device display in a calendar view. For example, the user interface may be configured to display the calendar view having multiple information fields corresponding to a subset of the extended duration (e.g., days, weeks, months). The calendar view may include information corresponding to at least a first category of a set of categories, as well as a summary indication for the first category over the extended duration. In some examples, the user interface may be configured to receive a user input to cycle or toggle to a second category of the set of categories, which may trigger a change in the multiple information fields and the summary indication. Accordingly, the user interface may facilitate the display of information corresponding to different categories of the set of categories, thereby supporting the display of various information to a user 102 even with a limited display size.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

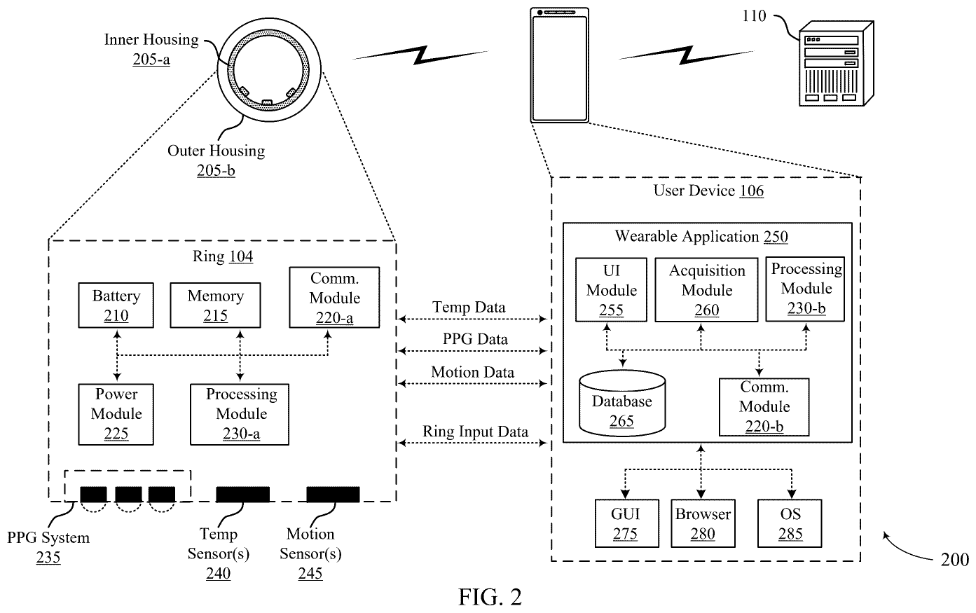

FIG. 2 illustrates an example of a system 200 that supports display of multiple data fields in a calendar view in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMl160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention”state) or otherwise generate an alert for the user.

In some aspects, the system 200 may support techniques for causing a user interface to display summary information corresponding to an extended duration via a portion of a device display in a calendar view. For example, one or more servers 110 or another device described herein may transmit one or more signals to cause a user interface of a user device 106 to display a calendar view. The calendar view may display information collected by the ring 104 (or another wearable device form factor as described herein) or based on physiological data collected by the ring 104, as described herein, for a subset of the extended duration. Additionally, or alternatively, the user interface may be configured to display summary information corresponding to a total for the extended duration, an average for the extended duration, or another summary metric, thereby displaying information that may be useful for a user within a portion of the calendar view. Additionally, the user interface may be configured to readily change (e.g., based on a user input) between a set of categories to change the displayed information, thereby facilitating the display of various types of information to a user.

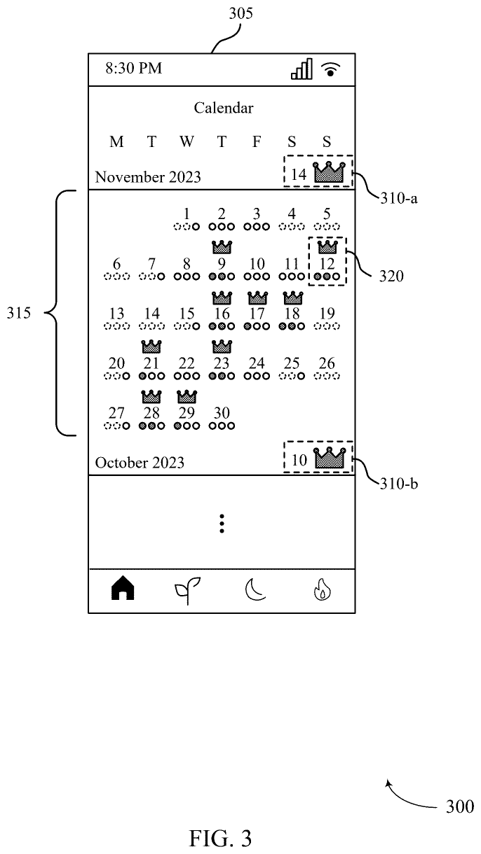

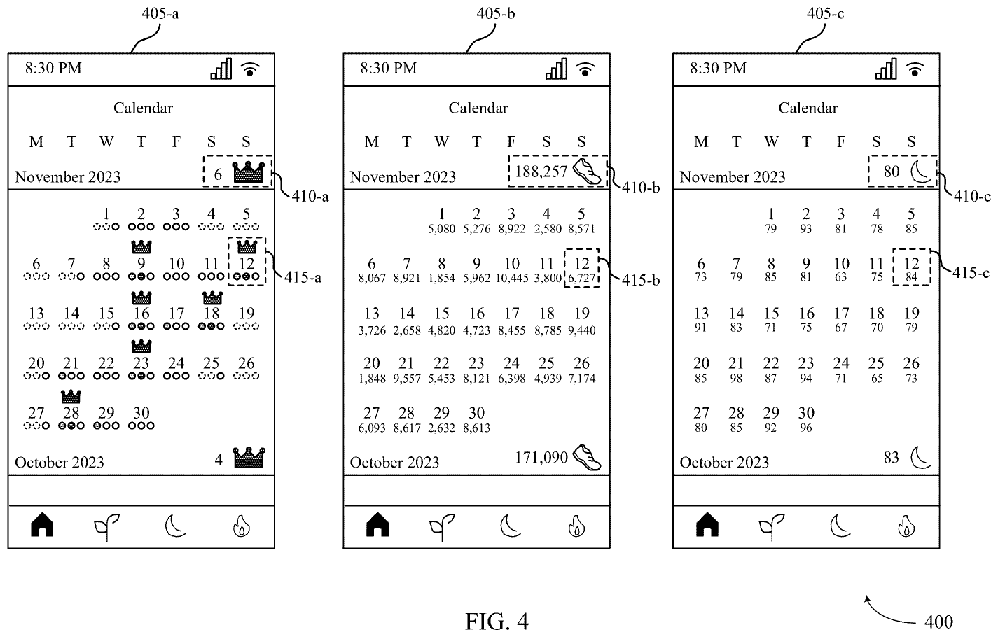

FIG. 3 shows an example of a GUI 300 that supports display of multiple data fields in a calendar view in accordance with aspects of the present disclosure. The GUI 300 may be implemented in a user device 106 or a wearable device 104, as described herein.

In some examples, one or more metrics may be determined based on physiological data collected from a user 102, as described herein. For example, one or more metrics may be determined (e.g., by one or more servers, by one or more machine learning classifiers, by the user device 106, by the wearable device 104, or some combination of these devices) based on the physiological data, such as a readiness metric, a sleep metric, and an activity metric. In some examples, the activity metric may provide a rating to the user 102 based on activity (e.g., exercise data, amount of time spent active) collected from the user 102, inactivity of the user 102, recovery periods, training frequency, training volume, achieving daily goals, and other metrics associated with an activity of the user 102.

The sleep metric may include a rating for the user 102 that characterizes the quality of sleep for the user 102. In some examples, the sleep metric may be determined based on a total amount of sleep, an efficiency of sleep (e.g., proportion of time sleeping while laying in bed), a restfulness of the user 102 (e.g., based on movement, heart measurements, temperature, or other measurements during sleep), time spent during various sleep stages (e.g., REM sleep, deep sleep, light sleep), latency of falling asleep (e.g., time to fall asleep), timing of sleep, or any combination thereof. The readiness metric may include a readiness score, which may be associated with a state of the body of the user 102 and whether more rest may be recommended to the user 102 (e.g., via the user device 106). In some examples, the readiness metric may be based on the sleep metric and the activity metric, on temperature, heart rate, heart rate variability, or other measurements collected from the user 102, or any combination thereof.

In some examples, the user 102 may be awarded a “crown” for a corresponding duration (e.g., a day) based on metrics measured from the user 102. For example, if the readiness metric, the activity metric, or the sleep metric for the user for the corresponding duration achieve a threshold value, a respective crown may be awarded to the user. Accordingly, the crowns may be able to indicate to the user 102 of a relative state of the user 102 based on the collected physiological data.

In accordance with examples as described herein, a user interface 305 may display information based on physiological data collected from the user 102 over a duration (e.g. a month). In some examples, the user interface 305 may display, via a portion of a calendar view, a summary indication 310. The summary indication 310 may correspond to a summary of a set of information fields 315 corresponding to the duration. For example, the set of information fields 315 may include information fields 320 corresponding to a portion of the duration (e.g., a day), and the summary indication 310 may indicate a summary (e.g., a sum, an average, a minimum or maximum) corresponding to each of the information fields 320 in the set of information fields 315.

In some examples, each information field 320 may indicate a quantity of crowns awarded (e.g., earned) by the user 102 based on the readiness metric, the activity metric, the sleep metric, or other metrics. Additionally, or alternatively, each information field 320 may indicate a relative value of the readiness metric, the activity metric, the sleep metric, or other metrics. For instance, each information metric 320 may include a circular indicator corresponding to a respective metric, and the circular indicator may indicate a relative value of each respective metric.

In some cases, a shading, color, or presence of the circular indicator may indicate a relative performance for the respective metric. In some examples, a non-shaded (e.g., non-colored, or an absent) circular indicator may indicate that a respective metric is below a first threshold, a lightly shaded (e.g., colored) circular indicator may indicate that the respective metric is between the first threshold and a second threshold, and a dark-shaded circular indicator may indicate that the respective metric is above the second threshold. In some cases, the second threshold may be associated with earning a crown for the respective metric. For example, a dark-shaded circular indicator may indicate that a crown has been earned for the respective metric.

In some examples, the information field 320 may display an icon to indicate whether at least one crown has been earned during the corresponding portion of the duration. For example, if at least one crown for the readiness metric, the sleep metric, or the activity metric has been awarded to the user 102 for November 12^{th}, a crown icon may be displayed on the information field 320 for the corresponding day. In some examples, the icon may also indicate a quantity of crowns earned for the portion of the duration. For example, a shading or color of the icon may depend on the quantity of crowns awarded to the user 102 for a given day.

In some examples, the user interface 305 may display a summary indication 310 for one or more durations on the user interface 305. For example, the user interface 305 may display a summary indication 310-a corresponding to a first duration (e.g., November) and a summary indication 310-b corresponding to a second duration (e.g., October). In some examples, the summary indication 310-a may display a value corresponding to a quantity of crowns awarded to the user 102 during the first duration. For example, the summary indication 310-a may correspond to a quantity of crowns for readiness, sleep, and activity awarded to the user 102 during the first duration.

Accordingly, by displaying summary indications 310 and information fields corresponding to various metrics determined based on physiological data collected from the user 102, multiple types of health and activity insights may be provided to the user 102 using a compact calendar view within a user interface 305.

FIG. 4 shows an example of a GUI 400 that supports display of multiple data fields in a calendar view in accordance with aspects of the present disclosure. The GUI 400 illustrates a user interface 405-a, a user interface 405-b, and a user interface 405-c that may be implemented in a user device 106 or a wearable device 104, as described herein.

In some examples, a user interface 405 may display a summary indication 410 and a plurality of information fields 415 corresponding to crowns awarded to a user 102, as described herein. For example, a summary indication 410 may display a total quantity of crowns awarded to the user based on a readiness metric, a sleep metric, and an activity metric. In some cases, however, a user 102 may desire to access information corresponding to a specific type of metric in a compact format, without having to access each respective information field 415 or count circular indicators. Additionally, or alternatively, the user 102 may be looking for information corresponding to specific metrics measured based on the physiological data collected from the user 102.

In accordance with examples as described herein, a user interface 405 may be configured to readily update a summary indication 410 and corresponding information fields 415 based on an input received from the user 102. In some examples, the user interface 405 may receive a user input, for example, at a portion of the user interface 405 associated with the summary indication 410. In some cases, the user input may be a press (e.g., tap) at the portion associated with the display of the summary indication 410. Additionally, or alternatively, the user input may be or include speech inputs indicating to change the category (e.g., via a microphone of the user device), via gestures (e.g., motions drawn a portion of the user interface 405), facial expressions or sign language (e.g., captured via a camera of a user device), via moving a slider displayed on the user interface 405 corresponding to the categories, or making a drawing on the user interface 505 (e.g., corresponding to a desired category).

In some examples, in response to the user input, a user interface 405-a may update a summary indication 410-a to display information associated with a quantity of crowns awarded to the user 102 based on a specific metric. For instance, the user interface 405-a may transition from displaying a total quantity of crowns earned during the duration (e.g., as illustrated in the GUI 300), to displaying a total quantity of crowns associated with a sleep metric (e.g., or an activity metric, or a readiness metric) earned during the duration. In some examples, an information field 415-a may update to highlight (e.g., using color or shading) a specific circular indicator, corresponding to the selected category of crown. In some cases, an icon included in the summary indication 410-a and the information field 415-a may change colors, shading, shape, or a combination thereof, to indicate the change in the displayed information. As such, the user interface 405-a may readily cycle between different categories of specific information to display to the user 102.

In some examples, in response to the user input, a user interface 405-b may update a summary indication 410-b to display a summary associated with a quantity of steps taken by the user 102, as measured by a wearable device 104 (e.g., based on physiological data collected by the wearable device 104). For example, the summary indication 410-b may display a total quantity of steps taken over the duration by the user 102, and the summary indication 420-b may display an icon to indicate that the information corresponds to steps taken. Additionally, or alternatively, the summary indication 410-b may display an average quantity of steps taken during each day of the duration. In some cases, the summary indication 410-b may be configured to toggle between the average and the total quantities based on a user input (e.g., a press on the display of the quantity, via application settings).

Additionally, or alternatively, in response to the user input, the user interface 405-b may update an information field 415-b to display a quantity of steps taken during a corresponding portion of the duration (e.g., during the corresponding day). As such, the user interface 405-b may readily update the information fields 415 and the summary indication 410-b to display information corresponding to steps taken, which may support the display of multiple information types on limited device displays.

In some examples, in response to the user input, a user interface 405-c may update a summary indication 410-b to display a summary associated with a sleep score for the user 102. For example, a sleep score may be determined based on physiological data collected from the user 102 while the user 102 sleeps, as described herein. In some examples, the summary indication 410-c may display an average sleep score for the duration (e.g., for the month). Additionally, the summary indication 410-c may be updated to display an icon indicating that the display information corresponds to sleep scores. In some cases, an information field 415-c may be updated to display a sleep score for a corresponding portion of the duration (e.g., for that day). Accordingly, the user interface 405-c may be readily updated to display information about sleep scores of the user 102, which may facilitate displaying sleep information for the user 102 over a large duration (e.g., over a month), such as when screen space is limited.

While the user interface 405-a, the user interface 405-b, and the user interface 405-c are described to illustrate crown information, step information, and sleep metric information, a user interface 405 may be configured to display various other additional types of information not shown. For example, the user interface 405 may be configured to display information corresponding to at least a first category of a set of categories, and cycle between the set of categories based on the received user input. For example, in some cases, an information field 415 may display information corresponding to multiple categories such as crowns for multiple different metrics (e.g., sleep crowns, activity crowns, readiness crowns), as illustrated by the user interface 305.

In some examples, in addition to information associated with crowns (e.g., total crowns, sleep crowns, activity crowns, readiness crowns), steps taken, and sleep scores, the set of categories may include heart rate information (e.g., minimum heart rate, maximum heart rate, average or resting heart rate, heart rate variability, heart health metrics), temperature information (e.g., maximum, minimum, or average temperature), caloric information (e.g., calories burned during exercise, activities, or throughout the day), distance traveled (e.g., in meters, kilometers, feet, miles), a quantity of workouts, a time spent performing activities (e.g., workouts), stress metrics, resilience metrics, blood information (e.g., blood oxygen, blood glucose), period information (e.g., days of a month where a period was experienced), metabolism information (e.g., based on physiological data collected from the user 102), or any combination thereof.

Additionally, or alternatively, the set of categories may include one or more categories associated with a classification of user activity. For example, one or more categories may correspond to a type of exercise or activity (e.g., running, biking, walking), which may be indicated by the icon displayed with the summary indication 410. In some examples, the summary indication 410, the information fields 415, or both, may correspond to a quantity of logged activities corresponding to the category, a total distance traveled during activities corresponding to the category, calories burned while performing activities corresponding to the category, or other information. In some cases, the classification of the user activity may be based on a predicted activity classification (e.g., using one or more machine learning models) using activity data collected from the user 102. Additionally, or alternatively, the classification of user activity data may be based on a received user input, such as an input of one or more activity tags (e.g., custom tags) for activity segments (e.g., exercises) performed by the user 102.

In some cases, the user input may cause a user interface 405 to cycle between categories of the set of categories. For example, in direct response to the user input, the user interface 405 may (e.g., automatically, immediately or relatively quickly) update the information fields 415 and the summary indications 410 from a first category to a second category of the set of categories. In some examples, the categories may cycle in a configured order, and the categories may repeat after the configured order is cycled through. Additionally, or alternatively, in direct response to the user input, the user interface 405 may display a list (e.g., a drop-down menu, a set of entries) including at least some of the set of categories. The user interface 405 may then update the information fields 415 and the summary indications 410 in response to a second user input to select a category displayed included in the list.

In some examples, each category may be associated with a type of summary indication 410. For example, the user interface 405 may display a type of summary indication 410 (e.g., best-fitting summary information), such as a sum (e.g., total), an average, a maximum or a minimum value, or other summary metrics, based on the category. For instance, a category corresponding to an amount of time spent performing activities (e.g., workouts) may be associated with the display of a sum for the summary indication 410, while a sleep score (e.g., and other “score” metrics) may be associated with the display of an average for the summary indication 410. In some cases, as a quantity of days varies for each month, displaying an average for the summary indication 410 may be most reliable, and may be displayed as a default (e.g., unless changed in request to a user input).

FIG. 5 shows an example of a GUI 500 that supports display of multiple data fields in a calendar view in accordance with aspects of the present disclosure. The GUI 500 illustrates an example user interface 505 that may be implemented in a user device 106 or a wearable device 104, as described herein.

In some cases, a user 102 may desire to view a summary of data for a larger or smaller duration than displayed in the user interface 505, such as data for a specific week or for a period of multiple months (e.g., quarterly) or a whole year. In accordance with examples as described herein, the user interface 505 may be configured to adapt a display based on a user input to display information with higher specificity, or a larger quantity of information.

In some examples, the user interface 505 may receive a user input, and the user interface 505 may adjust a quantity of information fields 520 displayed in response to the user input. In some cases, the user input may correspond to a pinching motion of the user interface 505. For example, an inward pinching motion (e.g., a zoom-in input) may trigger the user interface 505 to update the information displayed to correspond to a smaller duration (e.g., from a year to quarterly, from quarterly to a month, from a month to a week, from a week to a day), and adjust (e.g., reduce) the quantity of information fields 520 accordingly. Conversely, an outward pinching motion (e.g., a zoom-out input) may trigger the user interface 505 to update the information displayed to correspond to a larger duration, and adjust (e.g., increase) the quantity of information fields 520 accordingly.

In some examples, the information displayed by the user interface 505 may correspond to a position of the user input within the user interface 505. For example, a user input received at a position corresponding to a first week may cause the user interface 505 to display information for the first week.

In some examples, the user interface 505 may update values for summary indications 510, such as a summary indication 510-a, a summary indication 510-b, and a summary indication 510-c, in accordance with the updated duration. For example, the summary indication 510-a may display a total quantity of crowns awarded to the user 102 for a corresponding week. Additionally, a total quantity of each set of information fields 515 may be adjusted in accordance with the updated duration. For example, the set of information fields 515-a and the set of information fields 515-b may each have a quantity of information fields corresponding to a respective quantity of days for a corresponding week. In some cases, the user interface 505 may display a week number corresponding to the week of the year (e.g., as shown), or to a corresponding week of the month.

Additionally, or alternatively, the user interface 505 may adjust information displayed by each information field 520, such as an information field 520-a, an information field 520-b. For example, each information field 520 may display additional information based on the total quantity of information fields 520 being reduced, which may allow each information field 520 to cover a larger portion of the user interface 505. In some examples, the information field 520-a may display crown icons for each type of crown that may be awarded to the user 102. For instance, the information field 520-a may display three crown icons corresponding to three types of metrics (e.g., sleep, activity, and readiness), and each icon may indicate whether a corresponding crown was awarded to the user 102 for the corresponding duration (e.g., using color or shading).

In some examples, the user interface 505 may be configured to transition to a monthly calendar view (e.g., as illustrated by the user interface 305) based on receiving a user input (e.g., an outward pinch input). Additionally, or alternatively, the user interface 505 may be configured to transition to a daily calendar view based on receiving a different user input (e.g., an inward pinch input), and the daily calendar view may include additional information for each day. In some cases, the calendar view may transition based on other user inputs, such as speech inputs (e.g., via a digital assistant), via gestures (e.g., motions drawn on the screen), facial expressions or sign language (e.g., captured via a camera of a user device), via moving a slider displayed on the user interface 505, or making a drawing on the user interface 505. In some examples, receiving a user input at a location associated with a summary indication 510 may cause the user interface 505 to display information corresponding to a different category, as described with reference to FIG. 4, while remaining in the current calendar view (e.g., a weekly calendar view).

Accordingly, the user interface 505 may be configured to adjust a quantity of information displayed based on receiving user inputs, which may facilitate the display of various categories of information within a limited screen area, as described herein.

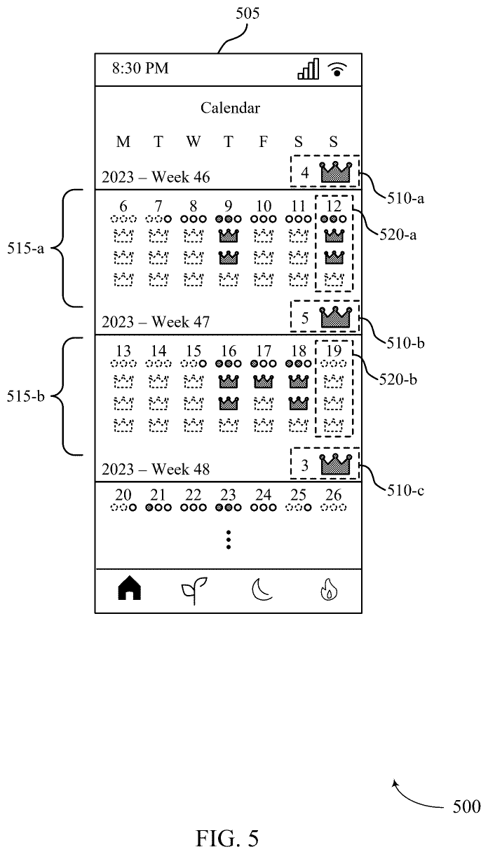

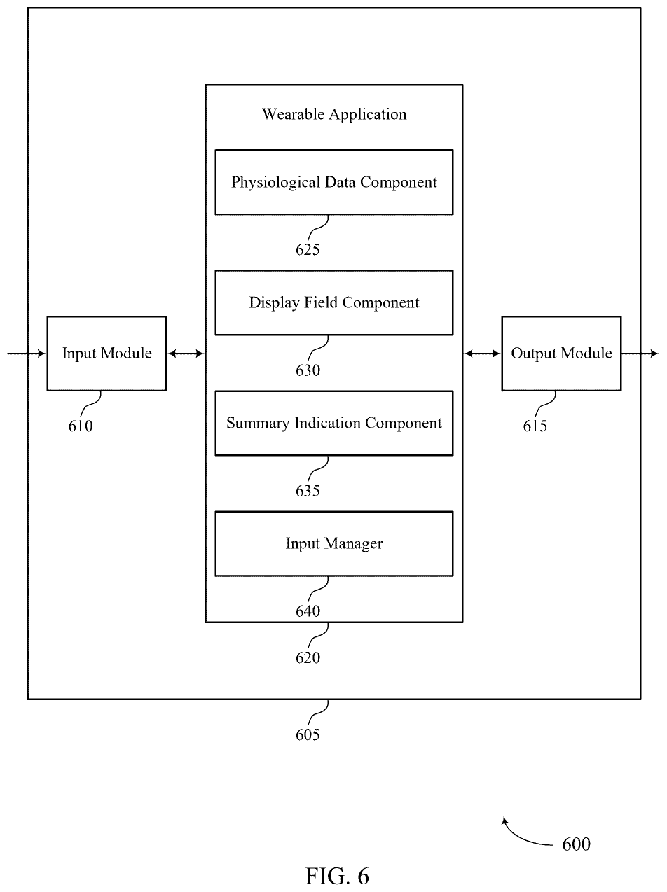

FIG. 6 shows a block diagram 600 of a device 605 that supports display of multiple data fields in a calendar view in accordance with aspects of the present disclosure. The device 605 may include an input module 610, an output module 615, and a wearable application 620. The device 605, or one of other components of the device 605 (e.g., the input module 610, the output module 615, the wearable application 620), may include at least one processor, which may be coupled with at least one memory, to support the described techniques. Each of these components may be in communication with one another (e.g., via one or more buses).

The input module 610 may provide a means for receiving information such as packets, user data, control information, or any combination thereof associated with various information channels (e.g., control channels, data channels, information channels related to illness detection techniques). Information may be passed on to other components of the device 605. The input module 610 may utilize a single antenna or a set of multiple antennas.

The output module 615 may provide a means for transmitting signals generated by other components of the device 605. For example, the output module 615 may transmit information such as packets, user data, control information, or any combination thereof associated with various information channels (e.g., control channels, data channels, information channels related to illness detection techniques). In some examples, the output module 615 may be co-located with the input module 610 in a transceiver module. The output module 615 may utilize a single antenna or a set of multiple antennas.

For example, the wearable application 620 may include a physiological data component 625, a display field component 630, a summary indication component 635, an input manager 640, or any combination thereof. In some examples, the wearable application 620, or various components thereof, may be configured to perform various operations (e.g., receiving, monitoring, transmitting) using or otherwise in cooperation with the input module 610, the output module 615, or both. For example, the wearable application 620 may receive information from the input module 610, send information to the output module 615, or be integrated in combination with the input module 610, the output module 615, or both to receive information, transmit information, or perform various other operations as described herein.

The physiological data component 625 may be configured as or otherwise support a means for receiving physiological data measured from a user via a wearable device. The display field component 630 may be configured as or otherwise support a means for displaying, via a user interface, a plurality of first display fields corresponding to a plurality of calendar fields, each of the plurality of calendar fields corresponding to a respective day of a month, wherein the plurality of calendar fields correspond to a subset of a plurality of preconfigured categories. The summary indication component 635 may be configured as or otherwise support a means for displaying a first summary indication associated with the plurality of first display fields, the first summary indication corresponding to at least a first category of the plurality of preconfigured categories and based on the physiological data. The input manager 640 may be configured as or otherwise support a means for receiving, via the user interface, a first input associated with a location of the first summary indication on the user interface, wherein the user interface is configured to toggle between the plurality of preconfigured categories based on the first input. The summary indication component 635 may be configured as or otherwise support a means for displaying, via the user interface, a second summary indication corresponding to a second category of the plurality of preconfigured categories based at least in part on receiving the first input, wherein the second summary indication replaces the first summary indication on the user interface.

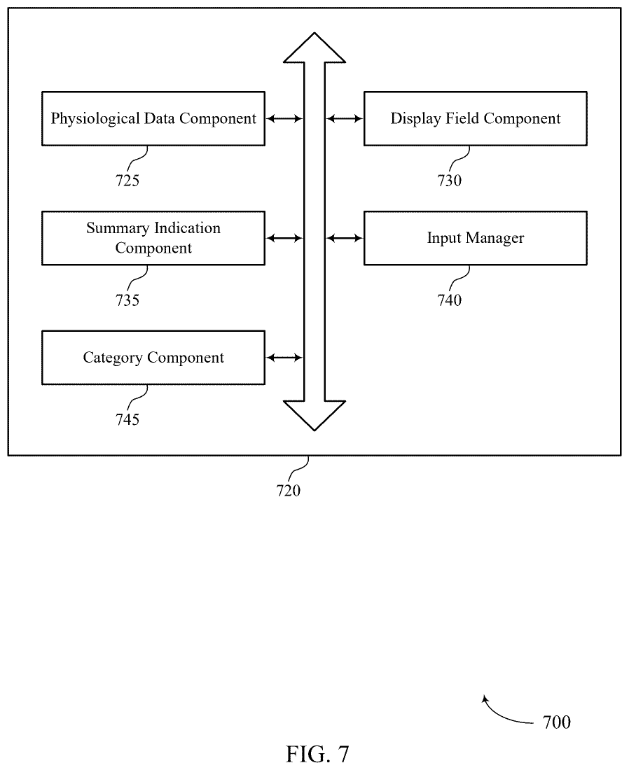

FIG. 7 shows a block diagram 700 of a wearable application 720 that supports display of multiple data fields in a calendar view in accordance with aspects of the present disclosure. The wearable application 720 may be an example of aspects of a wearable application or a wearable application 620, or both, as described herein. The wearable application 720, or various components thereof, may be an example of means for performing various aspects of display of multiple data fields in a calendar view as described herein. For example, the wearable application 720 may include a physiological data component 725, a display field component 730, a summary indication component 735, an input manager 740, a category component 745, or any combination thereof. Each of these components, or components of subcomponents thereof (e.g., one or more processors, one or more memories), may communicate, directly or indirectly, with one another (e.g., via one or more buses).

The physiological data component 725 may be configured as or otherwise support a means for receiving physiological data measured from a user via a wearable device. The display field component 730 may be configured as or otherwise support a means for displaying, via a user interface, a plurality of first display fields corresponding to a plurality of calendar fields, each of the plurality of calendar fields corresponding to a respective day, week, or month, wherein the plurality of calendar fields correspond to a subset of a plurality of preconfigured categories. The summary indication component 735 may be configured as or otherwise support a means for displaying a first summary indication associated with the plurality of first display fields, the first summary indication corresponding to at least a first category of the plurality of preconfigured categories and based on the physiological data. The input manager 740 may be configured as or otherwise support a means for receiving, via the user interface, a first input associated with a location of the first summary indication on the user interface, wherein the user interface is configured to toggle between the plurality of preconfigured categories based on the first input. In some examples, the summary indication component 735 may be configured as or otherwise support a means for displaying, via the user interface, a second summary indication corresponding to a second category of the plurality of preconfigured categories based at least in part on receiving the first input, wherein the second summary indication replaces the first summary indication on the user interface.

In some examples, the display field component 730 may be configured as or otherwise support a means for displaying, via the user interface, a plurality of second display fields corresponding to the plurality of calendar fields based at least in part on receiving the first input, wherein the plurality of second display fields replace the plurality of first display fields on the user interface.

In some examples, each display field of the plurality of second display fields corresponds to a respective value associated with the second category for the respective day, week, or month, and each respective value is based at least in part on the physiological data measured from the user.

In some examples, the second summary indication is based at least in part on a sum of each respective value corresponding to the plurality of second display fields, an average of each respective value corresponding to the plurality of second display fields, or both.

In some examples, the input manager 740 may be configured as or otherwise support a means for receiving, via the user interface, a second input associated with a location of the second summary indication on the user. In some examples, the summary indication component 735 may be configured as or otherwise support a means for displaying, via the user interface, a third summary indication corresponding to a third category of the plurality of preconfigured categories based at least in part on receiving the second input, wherein the third summary indication replaces the second summary indication on the user interface.

In some examples, the input manager 740 may be configured as or otherwise support a means for receiving, via the user interface, a third input associated with a location of the third summary indication on the user. In some examples, the summary indication component 735 may be configured as or otherwise support a means for displaying, via the user interface, the first summary indication based at least in part on receiving the third input, wherein the first summary indication replaces the third summary indication on the user interface.

In some examples, the category component 745 may be configured as or otherwise support a means for displaying, via the user interface, a set of entries in response to the first input, each entry of the set of entries corresponding to at least a second category of the plurality of preconfigured categories. In some examples, the input manager 740 may be configured as or otherwise support a means for receiving, via the user interface, a second input associated with a location of a first entry of the set of entries, wherein the second summary indication is displayed in response to the second input, and the second category corresponds to the first entry.

In some examples, the input manager 740 may be configured as or otherwise support a means for receiving, via the user interface, a second input associated with the plurality of calendar fields. In some examples, the display field component 730 may be configured as or otherwise support a means for updating a quantity of display fields shown via the user interface in response to the second input, wherein the quantity of display fields corresponds to a subset of the respective days, weeks, or months.

In some examples, the summary indication component 735 may be configured as or otherwise support a means for updating the first summary indication in accordance with the updated quantity of display fields based at least in part on receiving the second input.

In some examples, the plurality of preconfigured categories comprise an activity category, a readiness category, a sleep category, a heart rate category, a steps category, a calories burned category, a distance category, an activity tag category, a temperature category, a heart health category, a stress category, a resilience category, a period category, a blood oxygen category, a glucose category, a metabolism category, or a combination thereof.

In some examples, each display field of the plurality of first display fields corresponds to a respective value associated with the subset of the plurality of preconfigured categories for the respective day of the month, and each respective value associated with the first category is based at least in part on the physiological data measured from the user.

In some examples, the first summary indication is based at least in part on a sum of each respective value corresponding to the plurality of first display fields, an average of each respective value corresponding to the plurality of first display fields, or both.

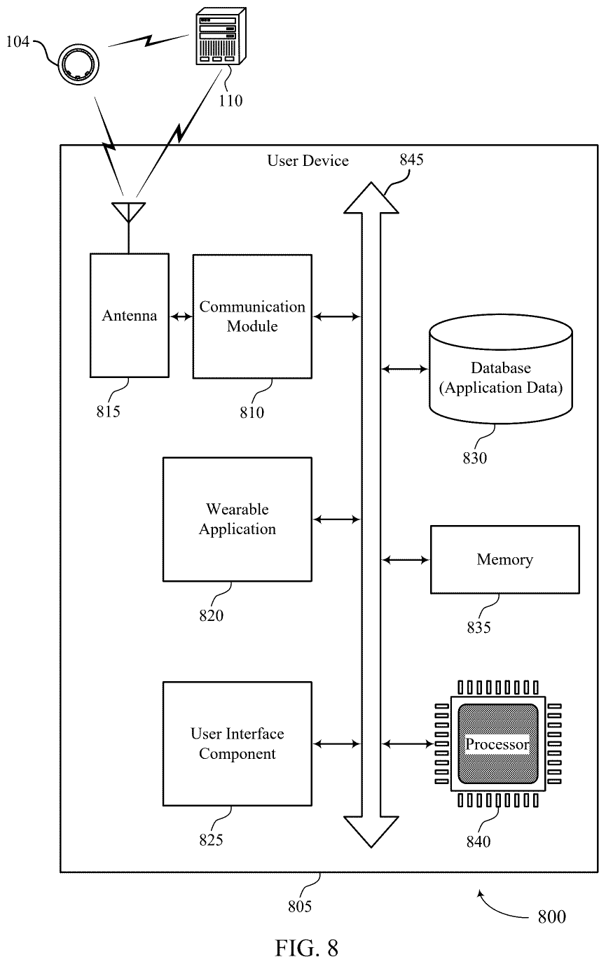

FIG. 8 shows a diagram of a system 800 including a device 805 that supports display of multiple data fields in a calendar view in accordance with aspects of the present disclosure. The device 805 may be an example of or include components of a device 605 as described herein. The device 805 may include an example of a user device 106, as described previously herein. The device 805 may include components for bi-directional communications including components for transmitting and receiving communications with a wearable device 104 and a server 110, such as a wearable application 820, a communication module 810, one or more antennas 815, a user interface component 825, a database (application data) 830, at least one memory 835, and at least one processor 840. These components may be in electronic communication or otherwise coupled (e.g., operatively, communicatively, functionally, electronically, electrically) via one or more buses (e.g., a bus 845).

The communication module 810 may manage input and output signals for the device 805 via the antenna 815. The communication module 810 may include an example of the communication module 220-b of the user device 106 shown and described in FIG. 2. In this regard, the communication module 810 may manage communications with the ring 104 and the server 110, as illustrated in FIG. 2. The communication module 810 may also manage peripherals not integrated into the device 805. In some cases, the communication module 810 may represent a physical connection or port to an external peripheral. In some cases, the communication module 810 may utilize an operating system such as iOS®, ANDROID®, MS-DOS®, MS-WINDOWS®, OS/2®, UNIX®, LINUX®, or another known operating system. In other cases, the communication module 810 may represent or interact with a wearable device (e.g., ring 104), modem, a keyboard, a mouse, a touchscreen, or a similar device. In some cases, the communication module 810 may be implemented as part of the processor 840. In some examples, a user may interact with the device 805 via the communication module 810, user interface component 825, or via hardware components controlled by the communication module 810.

In some cases, the device 805 may include a single antenna 815. However, in some other cases, the device 805 may have more than one antenna 815, which may be capable of concurrently transmitting or receiving multiple wireless transmissions. The communication module 810 may communicate bi-directionally, via the one or more antennas 815, wired, or wireless links as described herein. For example, the communication module 810 may represent a wireless transceiver and may communicate bi-directionally with another wireless transceiver. The communication module 810 may also include a modem to modulate the packets, to provide the modulated packets to one or more antennas 815 for transmission, and to demodulate packets received from the one or more antennas 815.

The user interface component 825 may manage data storage and processing in a database 830. In some cases, a user may interact with the user interface component 825. In other cases, the user interface component 825 may operate automatically without user interaction. The database 830 may be an example of a single database, a distributed database, multiple distributed databases, a data store, a data lake, or an emergency backup database.

The memory 835 may include RAM and ROM. The memory 835 may store computer-readable, computer-executable software including instructions that, when executed, cause the processor 840 to perform various functions described herein. In some cases, the memory 835 may contain, among other things, a BIOS which may control basic hardware or software operation such as the interaction with peripheral components or devices.

The processor 840 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 840 may be configured to operate a memory array using a memory controller. In other cases, a memory controller may be integrated into the processor 840. The processor 840 may be configured to execute computer-readable instructions stored in a memory 835 to perform various functions (e.g., functions or tasks supporting a method and system for sleep staging algorithms).

For example, the wearable application 820 may be configured as or otherwise support a means for receiving physiological data measured from a user via a wearable device. The wearable application 820 may be configured as or otherwise support a means for displaying, via a user interface, a plurality of first display fields corresponding to a plurality of calendar fields, each of the plurality of calendar fields corresponding to a respective day, week, or month, wherein the plurality of calendar fields correspond to a subset of a plurality of preconfigured categories. The wearable application 820 may be configured as or otherwise support a means for displaying a first summary indication associated with the plurality of first display fields, the first summary indication corresponding to at least a first category of the plurality of preconfigured categories and based on the physiological data. The wearable application 820 may be configured as or otherwise support a means for receiving, via the user interface, a first input associated with a location of the first summary indication on the user interface, wherein the user interface is configured to toggle between the plurality of preconfigured categories based on the first input. The wearable application 820 may be configured as or otherwise support a means for displaying, via the user interface, a second summary indication corresponding to a second category of the plurality of preconfigured categories based at least in part on receiving the first input, wherein the second summary indication replaces the first summary indication on the user interface.

By including or configuring the wearable application 820 in accordance with examples as described herein, the device 805 may support techniques for improved utilization of display and screen resources, enabling for the display of multiple data fields to a user within a display of the device 805, and thereby improving the user experience.

The wearable application 820 may include an application (e.g., “app”), program, software, or other component which is configured to facilitate communications with a ring 104, server 110, other user devices 106, and the like. For example, the wearable application 820 may include an application executable on a user device 106 which is configured to receive data (e.g., physiological data) from a ring 104, perform processing operations on the received data, transmit and receive data with the servers 110, and cause presentation of data to a user 102.

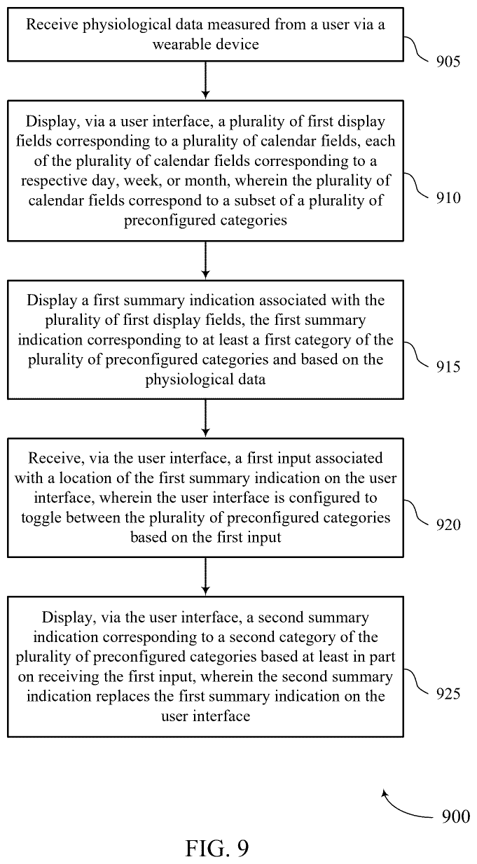

FIG. 9 shows a flowchart illustrating a method 900 that supports display of multiple data fields in a calendar view in accordance with aspects of the present disclosure. The operations of the method 900 may be implemented by a user device or its components as described herein. For example, the operations of the method 900 may be performed by a user device as described with reference to FIGS. 1 through 8. In some examples, a user device may execute a set of instructions to control the functional elements of the user device to perform the described functions. Additionally, or alternatively, the user device may perform aspects of the described functions using special-purpose hardware.

At 905, the method may include receiving physiological data measured from a user via a wearable device. The operations of 905 may be performed in accordance with examples as disclosed herein. In some examples, aspects of the operations of 905 may be performed by a physiological data component 725 as described with reference to FIG. 7.

At 910, the method may include displaying, via a user interface, a plurality of first display fields corresponding to a plurality of calendar fields, each of the plurality of calendar fields corresponding to a respective day of a month, wherein the plurality of calendar fields correspond to a subset of a plurality of preconfigured categories. The operations of 910 may be performed in accordance with examples as disclosed herein. In some examples, aspects of the operations of 910 may be performed by a display field component 730 as described with reference to FIG. 7.

At 915, the method may include displaying a first summary indication associated with the plurality of first display fields, the first summary indication corresponding to at least a first category of the plurality of preconfigured categories and based on the physiological data. The operations of 915 may be performed in accordance with examples as disclosed herein. In some examples, aspects of the operations of 915 may be performed by a summary indication component 735 as described with reference to FIG. 7.

At 920, the method may include receiving, via the user interface, a first input associated with a location of the first summary indication on the user interface, wherein the user interface is configured to toggle between the plurality of preconfigured categories based on the first input. The operations of 920 may be performed in accordance with examples as disclosed herein. In some examples, aspects of the operations of 920 may be performed by an input manager 740 as described with reference to FIG. 7.

At 925, the method may include displaying, via the user interface, a second summary indication corresponding to a second category of the plurality of preconfigured categories based at least in part on receiving the first input, wherein the second summary indication replaces the first summary indication on the user interface. The operations of 925 may be performed in accordance with examples as disclosed herein. In some examples, aspects of the operations of 925 may be performed by a summary indication component 735 as described with reference to FIG. 7.

It should be noted that the methods described above describe possible implementations, and that the operations and the steps may be rearranged or otherwise modified and that other implementations are possible. Furthermore, aspects from two or more of the methods may be combined.

A method by an apparatus is described. The method may include receiving physiological data measured from a user via a wearable device, displaying, via a user interface, a plurality of first display fields corresponding to a plurality of calendar fields, each of the plurality of calendar fields corresponding to a respective day, week, or month, wherein the plurality of calendar fields correspond to a subset of a plurality of preconfigured categories, displaying a first summary indication associated with the plurality of first display fields, the first summary indication corresponding to at least a first category of the plurality of preconfigured categories and based on the physiological data, receiving, via the user interface, a first input associated with a location of the first summary indication on the user interface, wherein the user interface is configured to toggle between the plurality of preconfigured categories based on the first input, and displaying, via the user interface, a second summary indication corresponding to a second category of the plurality of preconfigured categories based at least in part on receiving the first input, wherein the second summary indication replaces the first summary indication on the user interface.

An apparatus is described. The apparatus may include one or more memories storing processor-executable code, and one or more processors coupled with the one or more memories. The one or more processors may individually or collectively be operable to execute the code to cause the apparatus to receive physiological data measured from a user via a wearable device, display, via a user interface, a plurality of first display fields corresponding to a plurality of calendar fields, each of the plurality of calendar fields corresponding to a respective day, week, or month, wherein the plurality of calendar fields correspond to a subset of a plurality of preconfigured categories, display a first summary indication associated with the plurality of first display fields, the first summary indication corresponding to at least a first category of the plurality of preconfigured categories and based on the physiological data, receive, via the user interface, a first input associated with a location of the first summary indication on the user interface, wherein the user interface is configured to toggle between the plurality of preconfigured categories based on the first input, and display, via the user interface, a second summary indication corresponding to a second category of the plurality of preconfigured categories based at least in part on receiving the first input, wherein the second summary indication replaces the first summary indication on the user interface.

Another apparatus is described. The apparatus may include means for receiving physiological data measured from a user via a wearable device, means for displaying, via a user interface, a plurality of first display fields corresponding to a plurality of calendar fields, each of the plurality of calendar fields corresponding to a respective day, week, or month, wherein the plurality of calendar fields correspond to a subset of a plurality of preconfigured categories, means for displaying a first summary indication associated with the plurality of first display fields, the first summary indication corresponding to at least a first category of the plurality of preconfigured categories and based on the physiological data, means for receiving, via the user interface, a first input associated with a location of the first summary indication on the user interface, wherein the user interface is configured to toggle between the plurality of preconfigured categories based on the first input, and means for displaying, via the user interface, a second summary indication corresponding to a second category of the plurality of preconfigured categories based at least in part on receiving the first input, wherein the second summary indication replaces the first summary indication on the user interface.

A non-transitory computer-readable medium storing code is described. The code may include instructions executable by one or more processors to receive physiological data measured from a user via a wearable device, display, via a user interface, a plurality of first display fields corresponding to a plurality of calendar fields, each of the plurality of calendar fields corresponding to a respective day, week, or month, wherein the plurality of calendar fields correspond to a subset of a plurality of preconfigured categories, display a first summary indication associated with the plurality of first display fields, the first summary indication corresponding to at least a first category of the plurality of preconfigured categories and based on the physiological data, receive, via the user interface, a first input associated with a location of the first summary indication on the user interface, wherein the user interface is configured to toggle between the plurality of preconfigured categories based on the first input, and display, via the user interface, a second summary indication corresponding to a second category of the plurality of preconfigured categories based at least in part on receiving the first input, wherein the second summary indication replaces the first summary indication on the user interface.

Some examples of the method, apparatus, and non-transitory computer-readable medium described herein may further include operations, features, means, or instructions for displaying, via the user interface, a plurality of second display fields corresponding to the plurality of calendar fields based at least in part on receiving the first input, wherein the plurality of second display fields replace the plurality of first display fields on the user interface.

In some examples of the method, apparatus, and non-transitory computer-readable medium described herein, each display field of the plurality of second display fields corresponds to one or more respective values associated with the second category for the respective days, weeks, or months, and each of the one or more respective values may be based at least in part on the physiological data measured from the user.

In some examples of the method, apparatus, and non-transitory computer-readable medium described herein, the second summary indication may be based at least in part on a sum of each respective value corresponding to the plurality of second display fields, an average of each respective value corresponding to the plurality of second display fields, or both.

Some examples of the method, apparatus, and non-transitory computer-readable medium described herein may further include operations, features, means, or instructions for receiving, via the user interface, a second input associated with a location of the second summary indication on the user and displaying, via the user interface, a third summary indication corresponding to a third category of the plurality of preconfigured categories based at least in part on receiving the second input, wherein the third summary indication replaces the second summary indication on the user interface.

Some examples of the method, apparatus, and non-transitory computer-readable medium described herein may further include operations, features, means, or instructions for receiving, via the user interface, a third input associated with a location of the third summary indication on the user and displaying, via the user interface, the first summary indication based at least in part on receiving the third input, wherein the first summary indication replaces the third summary indication on the user interface.

Some examples of the method, apparatus, and non-transitory computer-readable medium described herein may further include operations, features, means, or instructions for displaying, via the user interface, a set of entries in response to the first input, each entry of the set of entries corresponding to category of the plurality of preconfigured categories and receiving, via the user interface, a second input associated with a location of a first entry of the set of entries, wherein the second summary indication may be displayed in response to the second input, and the second category corresponds to the first entry.

Some examples of the method, apparatus, and non-transitory computer-readable medium described herein may further include operations, features, means, or instructions for receiving, via the user interface, a second input associated with the plurality of calendar fields and updating a quantity of display fields shown via the user interface in response to the second input, wherein the quantity of display fields corresponds to a subset of the respective days, weeks, or months.

Some examples of the method, apparatus, and non-transitory computer-readable medium described herein may further include operations, features, means, or instructions for updating the first summary indication in accordance with the updated quantity of display fields based at least in part on receiving the second input.

In some examples of the method, apparatus, and non-transitory computer-readable medium described herein, the plurality of preconfigured categories comprise an activity category, a readiness category, a sleep category, a heart rate category, a steps category, a calories burned category, a distance category, an activity tag category, a temperature category, a heart health category, a stress category, a resilience category, a period category, a blood oxygen category, a glucose category, a metabolism category, or a combination thereof.

In some examples of the method, apparatus, and non-transitory computer-readable medium described herein, each display field of the plurality of first display fields corresponds to a respective value associated with the subset of the plurality of preconfigured categories for the respective day of the month, and each respective value associated with the first category may be based at least in part on the physiological data measured from the user.

In some examples of the method, apparatus, and non-transitory computer-readable medium described herein, the first summary indication may be based at least in part on a sum of each respective value corresponding to the plurality of first display fields, an average of each respective value corresponding to the plurality of first display fields, or both.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method, comprising:
receiving physiological data measured from a user via a wearable device;
displaying, via a user interface, a plurality of first display fields corresponding to a plurality of calendar fields, each of the plurality of calendar fields corresponding to a respective day, week, or month,, wherein the plurality of calendar fields correspond to a subset of a plurality of preconfigured categories;
displaying a first summary indication associated with the plurality of first display fields, the first summary indication corresponding to at least a first category of the plurality of preconfigured categories and based on the physiological data;
receiving, via the user interface, a first input associated with a location of the first summary indication on the user interface, wherein the user interface is configured to toggle between the plurality of preconfigured categories based on the first input; and
displaying, via the user interface, a second summary indication corresponding to a second category of the plurality of preconfigured categories based at least in part on receiving the first input, wherein the second summary indication replaces the first summary indication on the user interface.

2. The method of claim 1, further comprising:
displaying, via the user interface, a plurality of second display fields corresponding to the plurality of calendar fields based at least in part on receiving the first input, wherein the plurality of second display fields replace the plurality of first display fields on the user interface.

3. The method of claim 2, wherein each display field of the plurality of second display fields corresponds to a respective value associated with the second category for the respective day, week, or month, and each respective value is based at least in part on the physiological data measured from the user.

4. The method of claim 3, wherein the second summary indication is based at least in part on a sum of each respective value corresponding to the plurality of second display fields, an average of each respective value corresponding to the plurality of second display fields, or both.

5. The method of claim 1, further comprising:
receiving, via the user interface, a second input associated with a location of the second summary indication on the user; and
displaying, via the user interface, a third summary indication corresponding to a third category of the plurality of preconfigured categories based at least in part on receiving the second input, wherein the third summary indication replaces the second summary indication on the user interface.

6. The method of claim 5, further comprising:
receiving, via the user interface, a third input associated with a location of the third summary indication on the user; and
displaying, via the user interface, the first summary indication based at least in part on receiving the third input, wherein the first summary indication replaces the third summary indication on the user interface.

7. The method of claim 1, further comprising:
displaying, via the user interface, a set of entries in response to the first input, each entry of the set of entries corresponding to category of the plurality of preconfigured categories; and
receiving, via the user interface, a second input associated with a location of a first entry of the set of entries, wherein the second summary indication is displayed in response to the second input, and the second category corresponds to the first entry.

8. The method of claim 1, further comprising:
receiving, via the user interface, a second input associated with the plurality of calendar fields; and
updating a quantity of display fields shown via the user interface in response to the second input, wherein the quantity of display fields corresponds to a subset of the respective days, weeks, or months.

9. The method of claim 8, further comprising:
updating the first summary indication in accordance with the updated quantity of display fields based at least in part on receiving the second input.

10. The method of claim 1, wherein the plurality of preconfigured categories comprise an activity category, a readiness category, a sleep category, a heart rate category, a steps category, a calories burned category, a distance category, an activity tag category, a temperature category, a heart health category, a stress category, a resilience category, a period category, a blood oxygen category, a glucose category, a metabolism category, or a combination thereof.

11. The method of claim 1, wherein each display field of the plurality of first display fields corresponds to one or more respective values associated with the subset of the plurality of preconfigured categories for the respective days, weeks, or months,, and each of the one or more respective values associated with the first category is based at least in part on the physiological data measured from the user.

12. The method of claim 11, wherein the first summary indication is based at least in part on a sum of each respective value corresponding to the plurality of first display fields, an average of each respective value corresponding to the plurality of first display fields, or both.

13. An apparatus, comprising:
one or more memories storing processor-executable code; and
one or more processors coupled with the one or more memories and individually or collectively operable to execute the code to cause the apparatus to:
receive physiological data measured from a user via a wearable device;
display, via a user interface, a plurality of first display fields corresponding to a plurality of calendar fields, each of the plurality of calendar fields corresponding to a respective day, week, or month, wherein the plurality of calendar fields correspond to a subset of a plurality of preconfigured categories;
display a first summary indication associated with the plurality of first display fields, the first summary indication corresponding to at least a first category of the plurality of preconfigured categories and based on the physiological data;
receive, via the user interface, a first input associated with a location of the first summary indication on the user interface, wherein the user interface is configured to toggle between the plurality of preconfigured categories based on the first input; and
display, via the user interface, a second summary indication corresponding to a second category of the plurality of preconfigured categories based at least in part on receiving the first input, wherein the second summary indication replaces the first summary indication on the user interface.

14. The apparatus of claim 13, wherein the one or more processors are individually or collectively further operable to execute the code to cause the apparatus to:
display, via the user interface, a plurality of second display fields corresponding to the plurality of calendar fields based at least in part on receiving the first input, wherein the plurality of second display fields replace the plurality of first display fields on the user interface.

15. The apparatus of claim 14, wherein each display field of the plurality of second display fields corresponds to a respective value associated with the second category for the respective day, week, or month,, and each respective value is based at least in part on the physiological data measured from the user.

16. The apparatus of claim 15, wherein the second summary indication is based at least in part on a sum of each respective value corresponding to the plurality of second display fields, an average of each respective value corresponding to the plurality of second display fields, or both.

17. The apparatus of claim 13, wherein the one or more processors are individually or collectively further operable to execute the code to cause the apparatus to:
receive, via the user interface, a second input associated with a location of the second summary indication on the user; and
display, via the user interface, a third summary indication corresponding to a third category of the plurality of preconfigured categories based at least in part on receiving the second input, wherein the third summary indication replaces the second summary indication on the user interface.

18. The apparatus of claim 17, wherein the one or more processors are individually or collectively further operable to execute the code to cause the apparatus to:
receive, via the user interface, a third input associated with a location of the third summary indication on the user; and
display, via the user interface, the first summary indication based at least in part on receiving the third input, wherein the first summary indication replaces the third summary indication on the user interface.

19. The apparatus of claim 13, wherein the one or more processors are individually or collectively further operable to execute the code to cause the apparatus to:
display, via the user interface, a set of entries in response to the first input, each entry of the set of entries corresponding to category of the plurality of preconfigured categories; and
receive, via the user interface, a second input associated with a location of a first entry of the set of entries, wherein the second summary indication is displayed in response to the second input, and the second category corresponds to the first entry.

20. A non-transitory computer-readable medium storing code, the code comprising instructions executable by one or more processors to:
receive physiological data measured from a user via a wearable device;
display, via a user interface, a plurality of first display fields corresponding to a plurality of calendar fields, each of the plurality of calendar fields corresponding to a respective day, week, or month, wherein the plurality of calendar fields correspond to a subset of a plurality of preconfigured categories;
display a first summary indication associated with the plurality of first display fields, the first summary indication corresponding to at least a first category of the plurality of preconfigured categories and based on the physiological data;
receive, via the user interface, a first input associated with a location of the first summary indication on the user interface, wherein the user interface is configured to toggle between the plurality of preconfigured categories based on the first input; and
display, via the user interface, a second summary indication corresponding to a second category of the plurality of preconfigured categories based at least in part on receiving the first input, wherein the second summary indication replaces the first summary indication on the user interface.
